(19) United States
(12) Plant Patent Application Publication
(10) Pub. No.: US 2016/0205835 P1
(43) Pub. Date: Jul. 14, 2016

(54) Peach Tree White Delight Three

(50) ;

(71) Applicant: The Texas A&M University System, College Station, TX (US)

(72) Inventors: David H. Byrne, Bryan, TX (US); Natalie Anderson, Calvert, TX (US)

(73) Assignee: The Texas A&M University System, College Station, TX (US)

(21) Appl. No.: 14/544,523

(22) Filed: Jan. 13, 2015

(51) Int. Cl. A01H 5/00

(52) U.S. Cl. PLT/195

(57) ABSTRACT

Disclosed is a new variety of Prunus persica named ‘White Delight Three’. This new variety, which requires approximately 700 chilling units of dormancy, is considered to be a peach tree of mid to late season maturity, which produces subacid white-fleshed fruit that are firm, attractively colored, and suitable for both local and regional markets.

### BACKGROUND OF THE INVENTION

### Field of the Invention

This invention relates to peach trees and, more specifically, to peach trees referred to as a variety of Prunus persica named ‘WHITE DELIGHT THREE’ ‘WHITE DELIGHT THREE’, which requires approximately 700 chilling units of dormancy, produces an high quality, firm clingstone, subacid, white-fleshed peach that matures in the mid-season in the medium chill zone of Texas.

### SUMMARY OF THE INVENTION

The ‘WHITE DELIGHT THREE’ peach is characterized as to novelty and is otherwise noteworthy by producing fruit that ripens in mid season; is considered high quality; and which is firm and has an attractive coloration. In this regard, the present variety of peach tree bears fruit that are ripe for commercial harvesting and shipment in the last week of June and about 14 days after ‘Scarlet Pearl’ in the medium chill zone of Texas (Fairfield).

### ORIGIN OF THE VARIETY

The present peach tree was the result of an ongoing Stone Fruit Breeding Program of Texas A & M University, College Station, Brazos County, Tex. To this end, crosses are made each year to produce seedling populations from which improved progenies are evaluated and selected. ‘White Delight Three’ (WD3) tested as TX4F223LW resulted from a cross between a low chill, yellow fleshed peach selection, TXW1591-1, and a subacid, white nectarine developed in China, ‘Zao Hong Zhu’. TXW1591-1 is a yellow-fleshed, acid peach derived from a cross between ‘Tropicsweet’ and the Florida selection FLA9-6N (Fla6-8 open pollinated). ‘Zao Hong Zhu’ was released in 1994 by the breeding program of the Beijing Academy of Agriculture and Forest Science, Plant Protection and Environment Institute and was created by crossing the subacid, white-fleshed ‘Jingyu’ (=‘Okubo’×‘Okitsu’) and the Arkansas selection A369 (=7761 open pollinated) (Lirong Wang, personal communication). A369 is a large late ripening nectarine selected in Arkansas in 1986 and derived from materials developed in the New Jersey Fruit Breeding Program directed by Drs. Fred Hough and Catherine Bailey (John Clark, personal communication; Joseph Goffreda, personal communication). Resulting seed from this cross was planted in 1999 in a high density breeding orchard at the Texas A & M University Horticultural Farm in College Station, Tex. ‘WHITE DELIGHT THREE’ was marked as TX4F223LW for subsequent observation and noted as having exceptional characteristics. Two-year and older trees of the variety were subsequently evaluated during the 2006 through 2011 fruit growing seasons in both California (Fowler) and Texas (College Station, Fairfield, and Terrell).

### ASEXUAL REPRODUCTION OF THE VARIETY

‘WHITE DELIGHT THREE’ was bud grafted onto virus-free Nemaguard (“The Brooks and Olmo Register of Fruit and Nut Varieties,” 3rd Ed., American Society of Horticultural Science Press, Alexandria, Va., 1997) peach rootstock at the nursery site in Oakdale, Calif. The variety was subsequently planted at the experimental orchard in the central portion of the San Joaquin Valley, near Fowler, Fresno County, Calif. and in three sites in Texas (College Station, Fairfield and Terrell). Fruit from the resulting propagation has been evaluated during the period from 2006 to 2011 fruit seasons. This evaluation clearly demonstrated that the re-propagated trees were true to the characteristics of the original seedling in all observable aspects.

### BRIEF DESCRIPTION OF THE DRAWINGS

This new variety of peach tree is illustrated by the accompanying photographic drawings and depicts the plant by the best possible color representation using color photography, wherein:

FIG. 1. Color photograph of the fruit whole and cut both in axial and longitudinal axis showing the internal and external color and shape of the fruit. Fruit harvested on Jun. 21, 2007 in the experimental orchard in Fairfield, Tex.

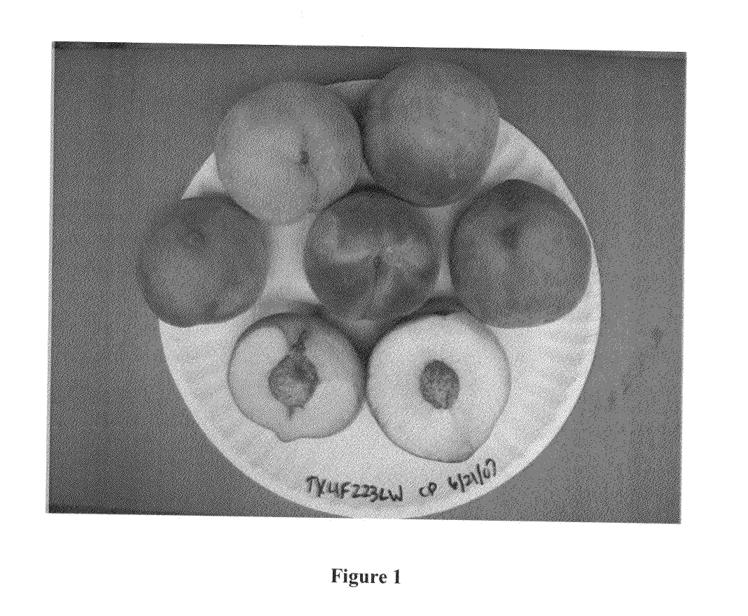

FIG. 2. Color photograph of ‘White Delight Three’ showing suture side and the fruit cut longitudinal axis showing the pit and flesh color.

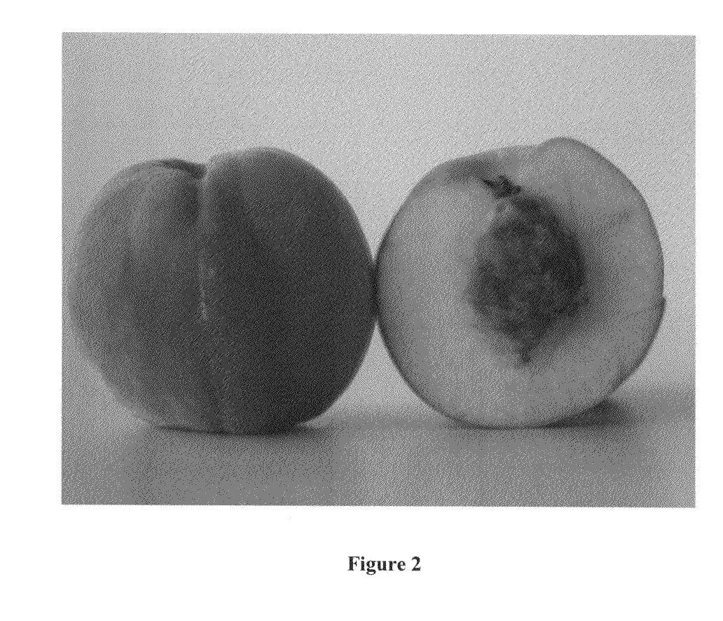

FIG. 3. Color photograph of the endocarp of ‘White Delight Three’ The ruler is demarcated in millimeters.

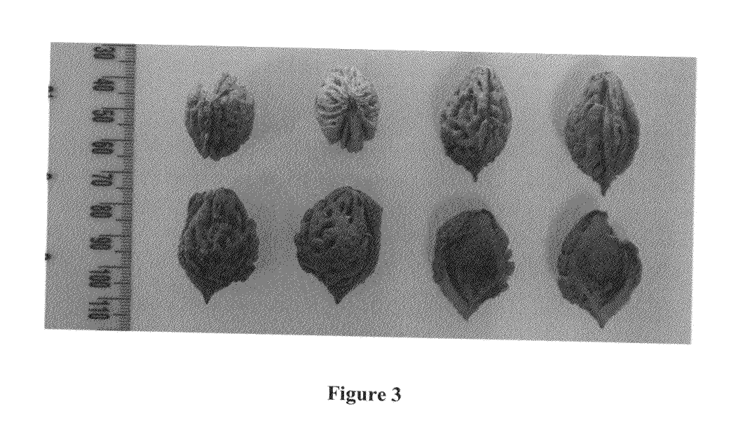

FIG. 4. A stem showing the leaves of the ‘White Delight Three’ peach. The ruler is demarcated in millimeters.

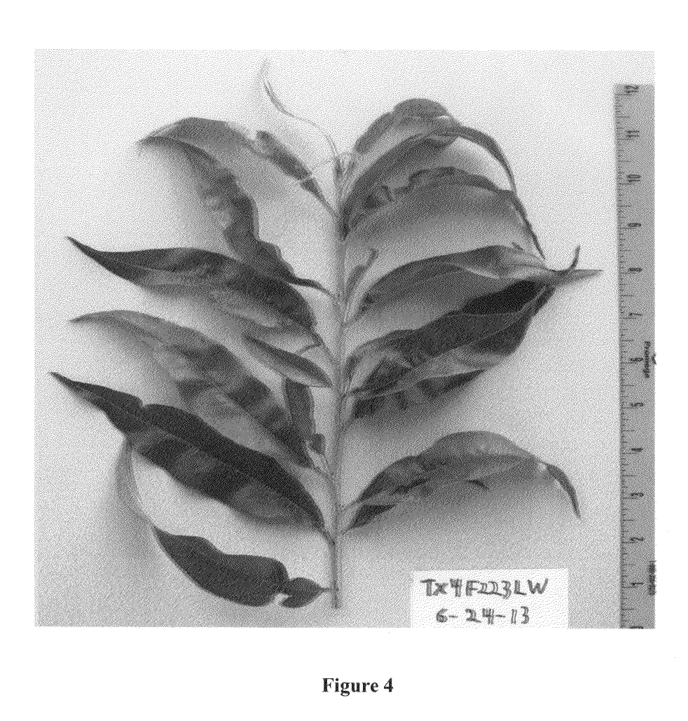

FIG. 5. Showy flowers of ‘White Delight Three’. The ruler is in millimeters.

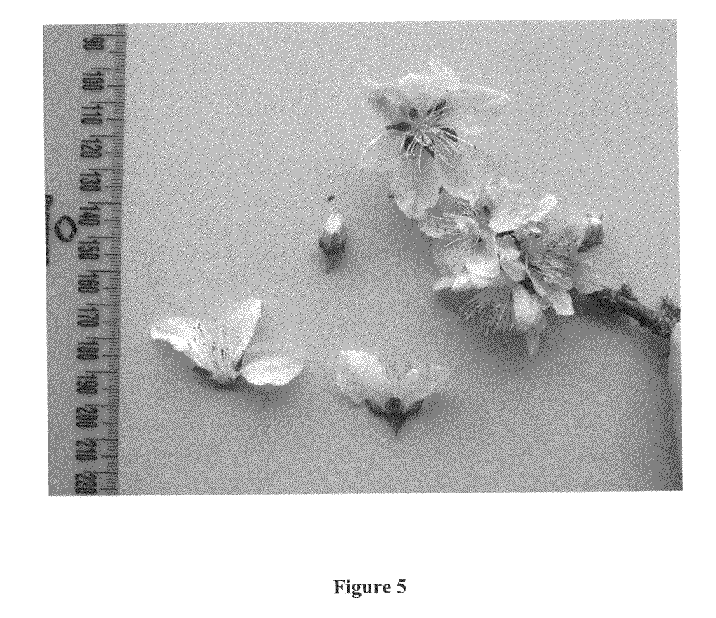

### BOTANICAL DESCRIPTION OF THE VARIETY

Referring more specifically to the pomological details of this new and distinct variety of peach tree, the following has been observed under the ecological conditions prevailing at the experimental orchards in the medium chill zone of Texas (Fairfield). All major color code designations are by reference to the R.H.S. Colour Chart (Third Edition) provided by The Royal Horticultural Society of Great Britain. Colors are approximate as color depends on horticultural practices such as light level and fertilization rate, among others.
Tree:
   Size.—Generally average as compared to other common peach cultivars ripening in the mid season of maturity.
   Height.—8 feet (2.44 m) on five-year old trees pruned to an open center training system.
   Width.—5 feet (1.52 m) on five-year old trees pruned to an open center training system.
   Vigor.—High.
   Density.—Medium to high.
   Productivity.—Productive.
   Shape.—The trees are vigorous with the typical semi-spreading growth habit similar to ‘TexKing’, ‘TexPrince’, and ‘TexRoyal’. No observations have been made on resistance for either peach rust or bacterial leaf spot.
   Current season growth.—The current season growth for the new variety was approximately 3.5 to 4.0 feet (1.1-1.2 m).
   Regularity of bearing.—Regular, and considered hardy under the typical climatic conditions of the medium chill zone of Texas and the central San Joaquin Valley, California.
Trunk:
   Size.—Approximately 3.8 inches (9.53 cm) in diameter and 13.5 inches (34.29 cm) in circumference when measured at a distance of approximately 12 inches (30.5 cm) above the soil level, at the end of the 2012 growing season on a five-year old tree.
   Bark texture.—Considered moderately rough with numerous folds of papery scarf-like skin being present.
   Bark coloration.—Variable, colors present are 198D of the Greyed-Green Group, 199C-D of the Greyed-Brown Group, N200D of the Brown Group and 201D of the Grey Group.
Branches:
   Size.—Considered medium for the species.
   Thickness.—Average (about 5.2 cm in diameter as measured 10 cm from the trunk on a five-year old tree) as compared to other varieties.
   Surface texture.—Average and appearing furrowed on wood that is several years old.
   Lenticels.—Numerous flat, oval lenticels present. The lenticels range in size from approximately 4 to 9 mm in width and were approximately 1 mm in height.
   Current season shoots.—Surface texture — Substantially glabrous.
   Internode length.—Approximately 3 to 5 cm as measured in the middle of a current season stem.
   Color of mature branches.—The predominant colors are 156A of the Greyed-White Group, 166A of the Greyed-Orange Group, 177A of the Greyed-Orange Group, N186C and 187A-B of the Greyed-Purple Group, 197C-D of the Greyed-Green Group and 199A of the Greyed-Brown Group.
   Current season shoots.—Color — Light green (144A-C, 145A, 146C-D of the Yellow-Green Group) with some reddish-brown coloration appearing on exposed surface of the shoots (166B-C, 172B-C, 173A and 174A-B of the Greyed-Orange Group).
Leaves:
   Size.—Considered average for the species. Leaf measurements have been taken from vigorous upright current season growth approximately at mid-shoot.
   Leaf length.—Approximately 170 to 185 mm.
   Leaf width.—Approximately 37 to 41 mm.
   Leaf thickness.—Less than 1 mm.
   Leaf form.—Lanceolate.
   Leaf tip form.—Acuminate.
   Leaf upper surface color.—Green varying among 137A-C of the Green Group and 146A-C of the Yellow-Green Group.
   Leaf lower surface color.—Green varying among 137C of the Green Group and 146B-D and 147A of the Yellow-Green Group.
   Leaf mid-vein color.—Light yellow green (N144C-D, 144B-D, 145C-D and 149D of the Yellow-Green Group).
   Leaf margins.—.
   Form.—Considered crenate.
   Uniformity.—Considered generally uniform.
   Leaf petioles.—.
   Size.—Considered medium long.
   Length.—Approximately 9 to 14 mm.
   Thickness.—Approximately 1 to 2 mm.
   Color.—Pale green (Yellow-Green Group 144A-B plus all of the colors of the leaf mid-vein).
   Leaf glands.—
   Size.—Approximately 1 to 2 mm in height and 1 to 1.5 mm in width.
   Number.—Generally 2 per leaf.
   Type.—Reniform.
   Color.—Brown with some green (199A, N199B of the Grey-Brown Group, 165A of the Greyed-Orange Group and 200D of the Brown Group).
   Leaf stipules.—.
   Size.—Small for the species.
   Length.—Approximately 2 to 3 mm.
   Form.—Lanceolate.
   Color.—Green (Yellow-Green 144B-C) with reddish brown tips (Greyed-Orange Group 166A-B and 175A-B) when young. The stipules are considered to be early deciduous.
   Ratio of wood (leaf) buds to flowering buds.—1 to 2 flower buds per vegetative bud.
Flowers:
   Floral buds.—.
   General.—The floral buds are considered to be medium to medium large in size, conic in form, and slightly appressed relative to the bearing shoot.
   Color.—The bud scales are gray-brown. The buds are considered hardy under climatic conditions of the medium chill zone of Texas and the central San Joaquin Valley, California.
   Length.—Average for species.
   Blooming type.—Considered medium in relation to other peach cultivars commonly growing in the medium chill zone of Texas and the central San Joaquin Valley. Date of full bloom was on average was about the 12th of March which was 2-4 days after ‘June Gold’ and 3-5 days before ‘Scarlet Pearl’.
   Flower type.—Showy.
   Flower size.—Flower diameter at full bloom is approximately 39 to 49 mm.
   Bloom quantity.—Considered abundant.
   Flower bud frequency.—Normally 1 to 2 per node.
   Petal size.—.
   General.—Considered medium to medium large for the species.
   Width.—Approximately 12 to 14 mm.
   Length.—Approximately 19 to 20 mm.
   Petal form.—Broadly ovate.
   Petal count.—Variable, from 5 to 7.
   Petal color.—Light pink (Red-Purple Group 69B-C and 73D).
   Petal claw.—.
   Form.—The claw is considered truncate in shape and has a medium size when compared to other varieties.
   Length.—Approximately 1 to 2 mm.
   Width.—Approximately 1 mm.
   Petal margins.—Generally considered variable, from nearly smooth to slightly undulate.
   Petal apex.—Generally — The petal apices appear slightly domed.
   Flower pedicel.—.
   Length.—Considered medium, and having an medium long length of approximately 4 to 5 mm.
   Thickness.—Considered average, approximately 1 mm.
   Color.—A light green (Yellow-Green Group 144D and N144A-D).
   Floral nectaries.—.
   Color.—Light yellow (Yellow Group 10A-B, 11B, 12C-D, 13D and Yellow-Orange Group 14D).
   Calyx.—.
   Surface texture.—Generally glabrous.
   Color.—Brownish red to maroon with green (Red-Purple Group 60A, Yellow-Green Group N144C-D, Greyed-Orange Group 176A, Greyed-Red Group 178A-B, Greyed-Purple Group 183A-B, 184A-B and 187C).
   Sepals.—.
   Surface texture.—The surface has a short, fine, wooly and a gray-colored texture.
   Size.—Average, and ovate in form.
   Color.—Brownish red to maroon with green (Red-Purple Group 60A, Yellow-Green Group N144C-D, Greyed-Orange Group 176A, Greyed-Red Group 178A-B, Greyed-Purple Group 183A-B, 184A-B and 187C).
   Anthers.—.
   General.—Average in size for the species.
   Color.—Brownish yellow (approximately Yellow-Orange Group 20A-B and Grey-Brown Group N199C).
   Pollen production.—Pollen is abundant, and is a yellow color.
   Filaments.—Size — Variable in length, approximately 13 to 14 mm, with the filaments generally equal to the pistil.
   Color.—Variable, from white to light pink (Red-Purple Group 69B-C, White Group 155A-D and N155B) and darkening with advanced maturity.
   Pistil.—.
   General.—Equal to the general anther height.
   Length.—Approximately 14 to 19 mm, including the ovary.
   Color.—Considered a very light green (Yellow-Green Group 145C-D), and becoming slightly darker with advancing senescence.
   Surface texture.—The variety has a long, silver white pubescent pistil (approximately White Group 155A-D).
   4.6 Fruit.—.
   Maturity when described.—The present variety of fruit is described, as it would be found in its firm ripe condition at full commercial maturity. Under the ecological conditions prevailing in the medium chill zone of Texas (Fairfield). ‘White Delight Three’ ripens at the end of June, about 14 days after ‘Scarlet Pearl’.
   Size.—General — Medium large for the season and considered uniform.
   Average cheek diameter.—Approximately 66 to 72 mm.
   Average suture diameter.—Approximately 68 to 72 mm.
   Average axial diameter.—Approximately 64 to 67 mm.
   Fruit form.—Generally round with some fruit having a small tip.
   Fruit suture.—Generally, the suture appears as a thin line that extends from the base to the apex, and appears deeper at the apex, forming a shallow basin at the apical point. No apparent callusing or stitching exists along the suture line.
   Color.—The suture is normally the same color as the underlying blush, however it has an additional medium red color (Red Group 39B).
   Ventral surface.—Form — Considered uniform.
   Stem cavity.—Size — Considered moderate for the species.
   Width.—Approximately 11 to 14 mm.
   Length.—Approximately 23 to 30 mm.
   Depth.—Approximately 15 to 20 mm.
   Fruit base.—Flat.
   Fruit apex.—Flat.
   Fruit stem.—Length — 9 to 13mm Thickness — 3 to 5 mm Color — Bright green with a greenish yellow coloration (Yellow-Green Group 144C and Yellow-Green Group N144A).
   Fruit skin.—Generally considered average in thickness. Surface Texture — Thin pubescence Skin Acidity — Considered neutral. Tenacious to Flesh — Yes at commercial maturity. Tendency to Crack — Not observed. Skin Color — Generally — Variable, with approximately 30-65% of the fruit surface covered with an attractive red blush. Down — Short and Thin Blush Color — blush color is generally more prevailing apically. This red blush ranges from a medium red (Orange-Red Group 34A) to a dark red (Orange-Red Group N34A) with many shades of blending and shading occurring between these colorations. Skin Ground Color — Very pale yellow with green (Yellow Green Group 154D).
   Flesh color.—Ivory (White Group 155A).
   Flesh fibers.—Present, numerous and lightly colored. These fibers are present throughout the flesh.
   Stone cavity color.—Very pale pink (White Group N155D).
   Flesh texture.—Generally, the flesh is considered firm and fine at commercial maturity.
   Ripening.—Generally the fruit of the present variety ripens evenly.
   Flavor.—Considered very sweet with a subacid flavor.
   Aroma.—Pleasant and reasonably abundant.
   Eating.—Generally considered very good.
Stone:
   Attachment.—Clingstone at commercial maturity.
   Stone size.—Generally considered medium relative to the ratio of stone to fruit size.
   Length.—Approximately 34 to 36 mm.
   Width.—Approximately 24 to 28 mm.
   Thickness.—Approximately 19 to 21 mm.
   Fibers.—Generally a few medium length fibers are attached along the entire surface of the stone.
   Stone form.—Generally variable, from ovate to obovate.
   Stone base angle.—The stone considered medium.
   Apex shape.—The stone apex is medium.
   Stone shape.—The stone normally ovid.
   Stone surface.—.
   Surface texture.—Minor surface markings are honeycombed with numerous single pits, chains of pits and pit grooves.
   Dorsal edge.—Shape — Grooved and having moderately rough edges.
   Stone color.—The color of the dry stone is light to medium brown (165C of the Greyed-Orange Group and N199C-D of the Grey-Brown Group). The color of the inside surface of the endocarp is primarily 164B-C and 165C of the Greyed-Orange Group.
   Tendency to split.—None.
   Kernel.—the kernel fills the endocarp at harvest and measures approximately 4-6 mm in thickness, 12-13 mm in width, and 17-20 mm in length. When dried the shriveled kernels measure approximately 3-4 mm in thickness, 12-13 mm in width, and 17-19 mm in length. The colors of the shriveled kernels are primarily of the Greyed-Orange Group 165B.
   Use.—The subject variety, ‘WHITE DELIGHT THREE’, is considered to be a peach tree of mid-season maturity, which produces fruit which are very firm, attractively colored and useful for the fresh market.
   Keeping quality.—Good.
   Resistance to insects and disease.—No particular susceptibilities were noted.
   Shipping quality.—Average. Although the new variety of peach tree possesses the described characteristics when grown under the ecological conditions prevailing near Fairfield in the medium chill zone of Texas, it will be understood that variations of the usual magnitude and characteristics incident to the changes in growing conditions, fertilization, pruning, and pest control are to be expected.

We claim:

1. A new and distinct Prunus persica tree, substantially as illustrated and described herein.
